(19) United States
(12) Patent Application Publication
(10) Pub. No.: US 2008/0058948 A1
(43) Pub. Date: Mar. 6, 2008

(54) Tricompartment prosthesis with reinforcement

(76) Inventors: Jean-Francois Biegun, Bavilliers (FR); Pascal Marceaux, Chaumont (FR)

Correspondence Address: Edward G. Greive;Renner, Kenner, Greive, Bobak, Taylor & Weber, Fourth Floor, First National Tower, Akron, OH, 44308-1456, US

(21) Appl. No.: 11/818,759

(22) Filed: Jun. 15, 2007

(30) Foreign Application Priority Data
Jun. 12, 2006 (FR) 06 05171

(51) Int. Cl. A61B 17/58; A61F 2/38

(52) U.S. Cl. 623/20.35; 606/62

(57) ABSTRACT

Femoral component of a knee prosthesis, comprising at least one, preferably two, condyles, on the posterior side; a trochlea part on the anterior side, in the upper surface of which a trochlea trajectory is defined; and a set of planar inner faces, intended to come into contact with corresponding resected faces of the end of a femur, defining an open inner cage on the inner surface of the component, the set of planar faces comprising a first planar anterior end face, a second planar intermediate face and a third planar posterior end face, characterised in that between the planar anterior end face and the planar intermediate face, a dome-shaped surface is formed having its concavity facing the outer side of the component.

### TECHNICAL FIELD

The present invention relates to a femoral component of a knee prosthesis and also to a knee prosthesis comprising a femoral component of this type.

### BACKGROUND ART

Numerous types of femoral components are known in the field of knee prostheses. These current femoral components generally comprise two condyles on the posterior side and a trochlea part on the anterior side, on the outer surface of which a trajectory for the trochlea is defined. The articulation of the knee is very complex. There are several movements in the articulation during flexion of the tibia, the femur rotating and sliding with respect to the tibial part, in the same way as the patella which slides and has a translatory movement on the trochlea. In order to ensure perfect kinematics of the patella on the femur while avoiding luxation phenomena, it is thus necessary that the trochlea trajectories can be well-defined in the trochlea part, and in particular the latter must be very hollow on its outer surface in order to define the trochlea trajectory. The prostheses must be thick so as to be able to accommodate such hollowing-out of the trochlea part. Besides, in the inner part of the femoral component, a set of planar faces define a cavity. In order to be able to realise a trochlea part in which a trochlea trajectory is sufficiently deeply defined, it is necessary today to ensure that this cavity comprises at least five planar faces. The combination of the necessary large thickness of the femoral component (thickness between its outer surface and the inner surface defined by the planar inner faces of the cavity), and by reason of having to provide five planar faces to form the inner cavity in order to be able to accommodate a trochlea trajectory which is very much hollowed out, means that, during the implantation of the prosthesis, the surgeon must resect much of the femoral bone. It is very harmful to withdraw a large amount of bone. The femur is weakened by this and during possible re-positioning of the prosthesis, the bone stock is generally no longer sufficient and it is thus necessary to use a very invasive implant, which necessitates a complex and costly operation.

### DISCLOSURE OF THE INVENTION

The present invention aims to overcome the drawbacks of the prostheses of the prior art by proposing a femoral component which, while being less thick, still allows a trochlea part which is as hollow as in the case of the prostheses of the prior art, in order to thus have a trochlea trajectory which extends as deeply in the outer surface of the prosthesis as in the case of prostheses of the prior art, in such a way that equally good kinematics of the prostheses are obtained at the same time as having to resect less bone during the implantation of the femoral component.

According to the invention, a femoral component of a knee prosthesis, comprising at least one, preferably two, condyles, on the posterior side; a trochlea part on the anterior side, in the upper surface of which a trochlea trajectory is defined; and a set of planar inner faces, intended to come into contact with corresponding resected faces of the end of a femur, defining an open inner cage on the inner surface of the component, the set of planar faces comprising a first planar anterior end face, so-called anterior cut, a second planar intermediate face, so-called distal cut, and a third planar posterior end face, so-called posterior cut, is characterised in that between the planar anterior end face and the planar intermediate face, a dome-shaped surface is formed with its concavity facing the outside of the component.

By thus providing this dome-shaped part between the anterior end part, that is to say essentially the trochlea part, and the intermediate face, it is thus possible to increase the trochlea trajectory which, after extending over the whole anterior end part, can continue along the dome-shaped surface. A trochlea trajectory is thus obtained which is as long as in the case of prostheses of the prior art and, however, it is no longer necessary to make a femoral component which is as thick as in the prior art, in which in particular it was necessary to provide an additional face between the anterior end face and the intermediate face.

According to an improvement of the invention, the dome-shaped surface is symmetrical in relation to the anteroposterior plane and the curve defining the cross section in the anteroposterior plane of this dome-shaped surface, at the level of the trochlea trajectory defined in the trochlea part, is in continuity with this same trochlea trajectory and in particular has the same radius of curvature and the same centre of curvature.

According to an improvement of the invention, the second planar intermediate face is at an angle of greater than 4°, preferably between 6° and 8°, in relation to the perpendicular to the first planar anterior end face.

According to an improvement of the invention, the second planar intermediate face is at an angle of greater than 4°, preferably between 6° and 8°, in relation to the perpendicular to the third planar posterior end face.

According to an improvement of the invention, the second planar intermediate face forms corners with each of the first and third planar anterior and posterior end faces, being contiguous therewith.

According to an improvement of the invention, the dome-shaped surface which projects inside the cavity has, in section in the mediolateral plane passing through the first planar anterior end face, the form of a curve which is included strictly inside the inner cavity, in such a way that two straight segments on either side of this dome-shaped surface form corner edge elements between the intermediate face and the anterior end face.

According to a preferred embodiment of the invention, reinforcements are provided in the corners formed between the third posterior end face(s) and the second intermediate face of the reinforcements.

The present invention also relates to a knee prosthesis comprising a femoral component according to the invention and a tibial part, a tibial insert being arranged between the tibial part and the femoral component.

### BRIEF DESCRIPTION OF THE DRAWINGS

An embodiment of the invention will now be described by way of example with reference to the drawing, in which:

FIG. 1 is a perspective view from above of a femoral component according to the invention,

FIG. 2 is a perspective view at a different angle of the femoral component of FIG. 1,

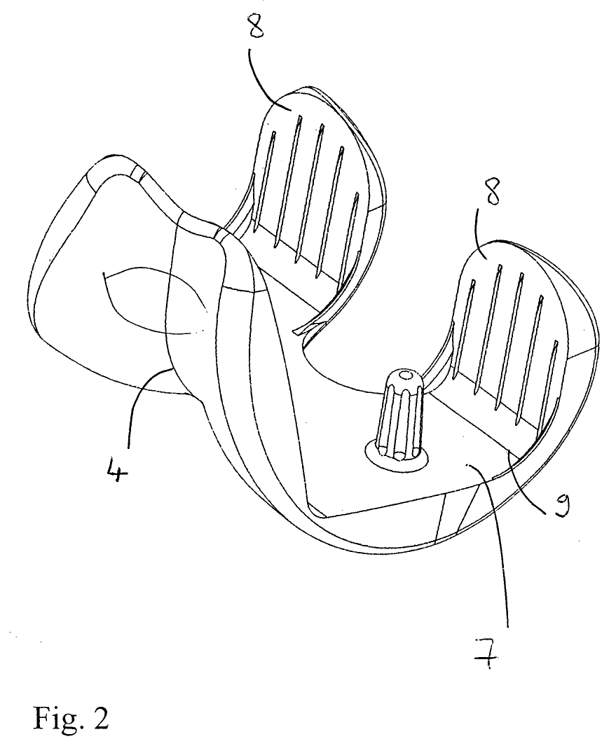

FIG. 3 is a side view of the femoral component of FIGS. 1 and 2,

FIG. 4 is a top view of the femoral component of FIGS. 1 to 3,

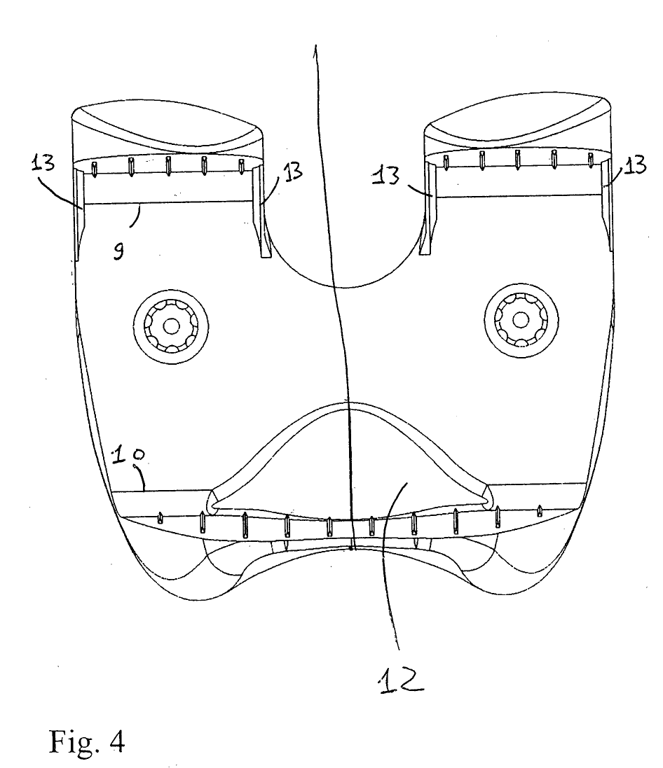

FIG. 5 is a face-on view of the femoral component of FIGS. 1 to 4.

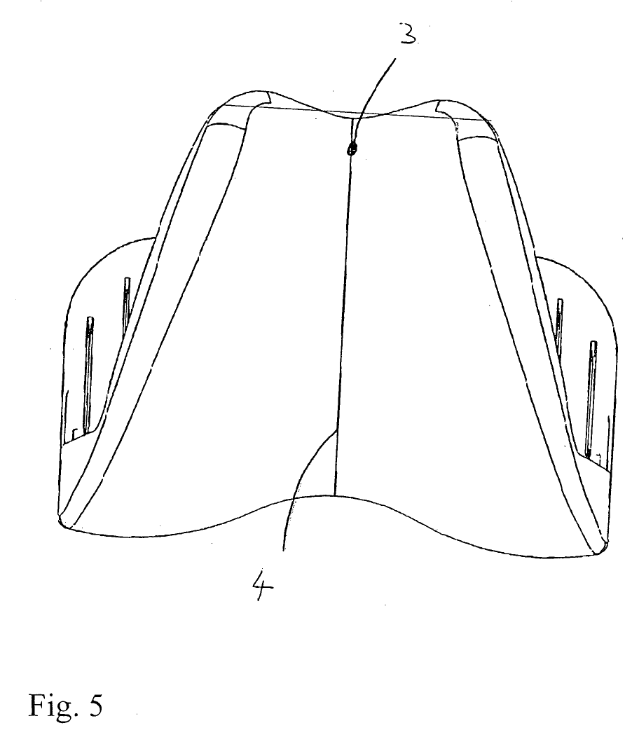

### PREFERRED EMBODIMENT FOR CARRYING-OUT THE INVENTION

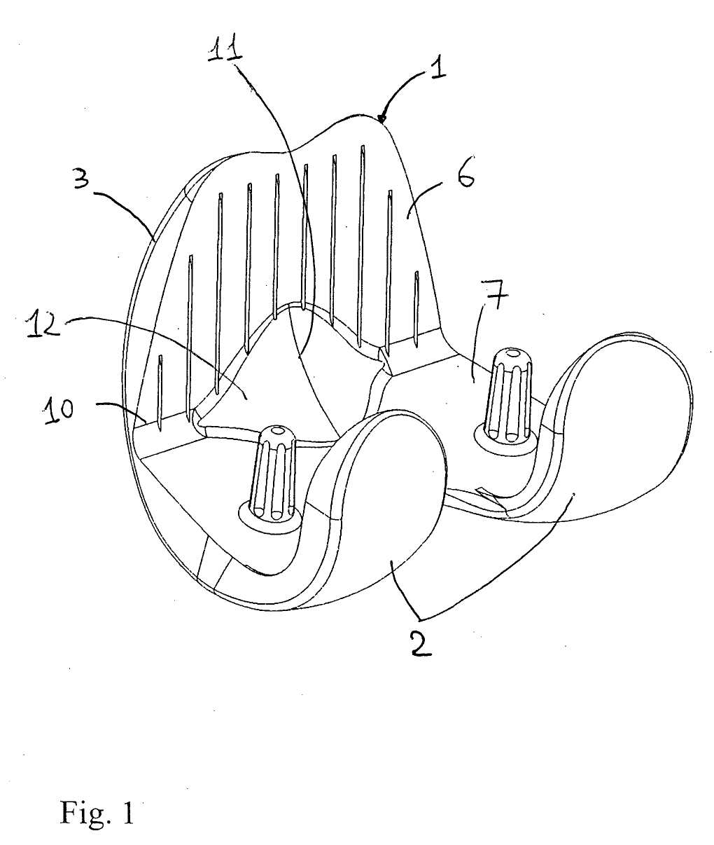

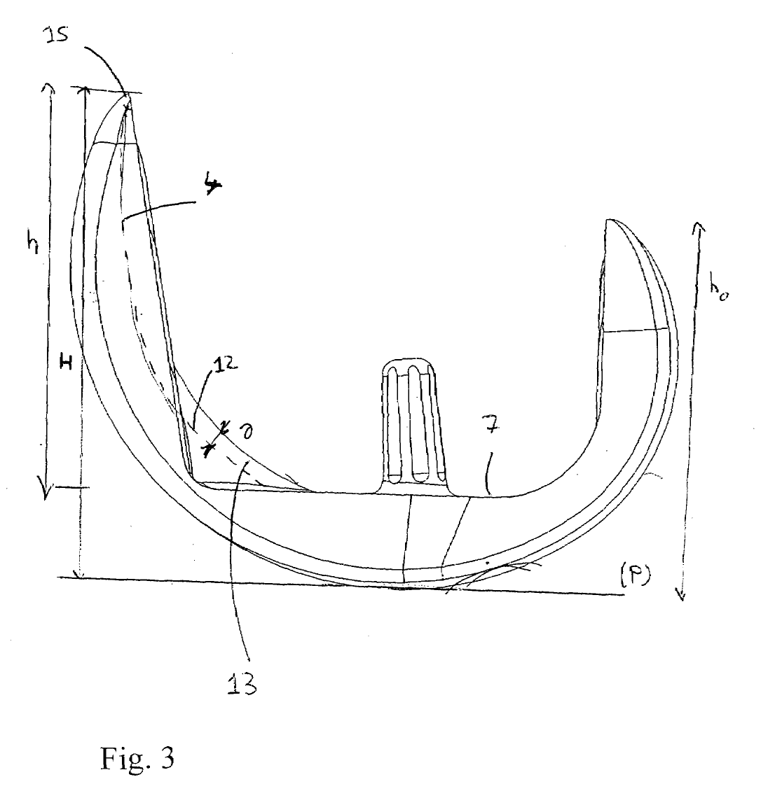

FIG. 1 shows a femoral component of a knee prosthesis 1 comprising two condyles 2 and a trochlea part 3. The two condyles in the anteroposterior plane (plane of FIG. 3) are defined essentially by a circular curve, in the same way as the trochlea part is defined by another circular part.

The trochlea part 3 in its outer part defines a trochlea trajectory 4. This trochlea trajectory is formed by a groove hollowed out in the outer surface of the trochlea part 3. The inner part of the femoral component defines a cavity having essentially planar faces. A first planar anterior end face 6 is thus defined, corresponding to the anterior cut of the femur, a second intermediate face 7, corresponding to the distal cut, and a third posterior end face 8, corresponding to the posterior cut of the femur. Two attachment points to the bone of the femur on which the femoral component is to be implanted project from the intermediate face 7. The intermediate face 7 is essentially perpendicular to the face 8 and the face 6. This inner cavity only comprises these three planar faces. Thus, the intermediate face 7 is essentially contiguous with the anterior end face 6 and the posterior end face 8. An edge element 9 defines the interface between the intermediate face 7 and the posterior end face 8. An edge element 10 defines the interface between the intermediate face 7 and the anterior end face 6.

Between the anterior end face 6 and the intermediate face 7, there is a dome-shaped surface 12, of which the convexity faces the inside of the cavity and the concavity the outside of the component. In cross section in the mediolateral plane, that is to say the plane perpendicular to the anteroposterior axis and passing via the axis of symmetry of the prosthesis, this dome-shaped surface 12 has the form of a bell. The set of tops of the set of bells of each cross section defines a top curve 11 of this dome-shaped surface 12. This curve 11 of the different tops has a concavity facing the inside of the prosthesis. It is the continuation of the trochlea trajectory defined in the trochlea part 3. In particular, the trochlea trajectory 4 has a radius of curvature identical to that of this curve 11 of the tops of the bells.

In cross section in the plane corresponding to the anterior end face 6, the dome-shaped surface extends strictly inside the inner cavity, in such a way that on each side of this surface 12 it is possible to define straight segments corresponding to the edge element 10 forming the interface between the anterior end face 6 and the intermediate face 7.

According to an improvement of the invention it is also possible to provide reinforcements 13 arranged in the corner between the posterior end face 8 and the intermediate face 7. In particular, these can be inserts in the form of a wall having an upper concave edge, of which the concavity faces the inside of the prosthesis. They allow the angles to be “rounded” between the intermediate face 7 and the posterior end faces 8 in order to avoid a break linked to the small thickness of the prosthesis as a whole. In fact, now that there are only three faces for forming the inner cavity, it is possible to make the prosthesis less thick because it has been possible, however, owing to the dome-shaped surface 11, to obtain a trochlea trajectory which is as long as in the prior art. In order to avoid an untimely break of the prosthesis, reinforcement elements 13 are thus provided at the corner between the posterior end face(s) and the intermediate face 7. Besides, so that these inserts do not necessitate the resection of receiving slots in the bone of the femur, said inserts are arranged laterally at the mediolateral extremities of the corners.

A reference plane P is defined which is parallel to the intermediate face and tangential to the outer surface of the femoral component. The height H of the femoral component is defined as being the distance between the furthest point 15 of the posterior end part and the plane P measured along a line passing via the end face 6, and the height h of the end face 6 the same distance measured as far as the intersection with the Intermediate face 7. The ratio

$$\frac{h}{H}$$

is greater than or equal to 0.75.

The height hₒ of the posterior end face is also defined parallel to this same face as far as the intersection with the intermediate face 7.

$$\frac{{h}_{0}}{h}$$

is preferably □ 0.6.

1. Femoral component of a knee prosthesis, comprising at least one condyles (2), on the posterior side; a trochlea part (3) on the anterior side, in the upper surface of which a trochlea trajectory (4) is defined; and a set of planar inner faces intended to come into contact with corresponding resected faces of the end of a femur, defining an open inner cage on the inner surface of the component, all the planar faces comprising a first planar anterior end face (6), a second planar intermediate face (7) and a third planar posterior end face (8), characterised in that between the planar anterior end face and the planar intermediate face, a dome-shaped surface (12) is formed with its concavity facing the outside of the component.

2. Component according to claim 1, characterised in that the dome-shaped surface is symmetrical in relation to the anteroposterior plane and the curve defining the cross section in the anteroposterior plane of this dome-shaped surface, at the level of the trochlea trajectory defined in the trochlea part, is in continuity with this same trochlea trajectory, and in particular has the same radius of curvature and the same centre of curvature.

3. Component according to claim 1, characterised in that the second planar intermediate face is at an angle of at least 4° in relation to the perpendicular to the first planar anterior end face.

4. Component according to claim 1, characterised in that the second planar intermediate face is at an angle of at least 4° in relation to the perpendicular to the third planar posterior end face.

5. Component according to claim 1, characterised in that the second planar intermediate face forms corners with each of the first and third planar anterior and posterior end faces, being contiguous therewith.

6. Component according to claim 1, characterised in that the dome-shaped surface projecting inside the cavity has, in section in the mediolateral plane passing via the plane of the first planar anterior end face, the form of a curve which is included strictly inside the interior cavity in such a way that two straight segments on either side of this dome-shaped surface form corner edge elements between the intermediate face and the anterior end face.

7. Component according to claim 1, characterised in that reinforcements are arranged in the corners formed between the third posterior end face(s) and the second intermediate face.

8. Component according to claim 1, characterised in that
$$\frac{h}{H} □ 0.75 .$$

9. Component according to claim 1, characterised in that
$$\frac{{h}_{0}}{h} □ 0.6 .$$

10. Knee prosthesis comprising a femoral component according to claim 1, and a tibial part, a tibial insert being arranged between the tibial part and the femoral component.
